(19) United States
(12) Patent Application Publication
(10) Pub. No.: US 2007/0048375 A1
(43) Pub. Date: Mar. 1, 2007

(54) Effervescent preparation of a basic medicinally active substance

(76) Inventors: Wolfgang Wiehl, Koln (DE); Ralf Bullesbach, Koln (DE); Dieter Rehn, Basel (CH); Irene Loose, Langenfeld (DE)

Correspondence Address: Richard S. Bullitt;Bayer HealthCare LLC, 36 Columbia Road, Morristown, NJ, 07962, US

(21) Appl. No.: 11/454,462

(22) Filed: Jun. 16, 2006

(30) Foreign Application Priority Data
Dec. 7, 2004 (WO) WO 2005/063199
Dec. 19, 2003 (DE) 103 59 790.5

(51) Int. Cl. A61K 9/46; A61K 31/60; A61K 31/198; A61K 31/192; A61K 31/137

(52) U.S. Cl. 424/466; 514/570; 514/165; 514/649; 514/562

(57) ABSTRACT

The present invention relates to an effervescent preparation comprising an effervescent composition, at least one basic medicinally active substance and, where appropriate, one or more further active ingredients, characterized in that alkalization of the urine and thus increased accumulation of the basic medicinally active substance is avoided, to medicaments comprising this effervescent preparation, to processes for the production thereof and to the use thereof.

The present invention relates to an effervescent preparation comprising at least one basic medicinally active substance, characterized in that alkalization of the urine and thus increased accumulation of the basic medicinally active substance is avoided.

Effervescent preparations as medicaments have been known for a long time and are used in the form of effervescent powders, tablets or granules. They are as a rule solid preparations comprising an acid/base pair which react on contact with water to evolve CO₂ (EP 0 474 040, EP 0 369 228, P. C. Schmidt, I. Christin, Die Pharmazie, 1990, 45, 89-110). The effervescent formulation leads to faster disintegration of the dosage form and dissolution of the active ingredient and may thus lead to better and faster bioavailability of the medicament than a conventional tablet formulation. For active ingredients with a long absorption time or with a tendency to irritate the gastric mucosa, for example, effervescent preparations are a formulation form able to alleviate the said disadvantageous properties of the active ingredient. Medicaments comprising effervescent preparations are therefore becoming increasingly popular.

When the advantages of an effervescent preparation were carried over to basic medicaments, it was found in a study (see below: study of the accumulation behaviour of pseudoephedrine) that on multiple administration of an effervescent preparation comprising for example pseudoephedrine as basic medicinally active substance using a conventional effervescent composition comprising sodium bicarbonate as basic component and citric acid as acidic component there is accumulation of the basic medicinally active substance in the patient which goes beyond the intrinsic accumulation behaviour in non-effervescent formulations. Optimal therapy is no longer ensured thereby, and there is an increasing risk of side effects occurring.

Under physiological conditions, basic medicaments are substantially in ionized form and renal excretion thereof is therefore sufficiently fast. Since the use of conventional effervescent formulations comprising bicarbonates or carbonates of singly charged cations such as, for example, sodium as base, and citric acid as acid, leads to alkalization of the urine, more of the basic medicaments is in the nonionized form. The elimination half-life is increased and the basic active ingredient accumulates in the systemic circulation, possibly leading to an altered benefit/risk assessment of the basic medicament. The strong dependence of the elimination half-life of basic active ingredients, for example of pseudoephedrine, on the pH of the urine is verified in the literature (I. Kanfer et al. Pharmacotherapy 1993, 13, 116S-128S).

The object was to find an effervescent formulation for basic active ingredients which avoids alkalization of the urine and thus accumulation of the basic active ingredient, and hence ensures optimal therapy and can reduce the risk of side effects occurring.

It has now been found, surprisingly, that alkalization of the urine and thus increased accumulation of the basic medicinally active substance going far beyond the intrinsic accumulation behaviour in non-effervescent formulations is avoided through the effervescent preparation according to the invention.

The present invention relates to an effervescent preparation comprising an effervescent composition, at least one basic medicinally active substance and, where appropriate, one or more further active ingredients, characterized in that increased accumulation of the basic medicinally active substance is avoided.

Basic medicinally active substances for the purposes of the invention have a protonatable basic primary, secondary or tertiary nitrogen such as, for example, pseudoephedrine, chlorphenamine, phenylephrine, diphenhydramine, clemastine, bromphenamine, hydroxyzine, tripelenamine, pyrilamine, mequitazine, promethazine, cyproheptadine, doxylamine, dexchlorphenamine, epinephrine, phenamine, trimeprazine, cyclizine, meclizine and azatadine, and the physiologically acceptable salts thereof. Pseudoephedrine, chlorphenamine, phenylephrine and clemastine, and the physiologically acceptable salts thereof, are preferred. Pseudoephedrine and its physiologically acceptable salts such as, for example, pseudoephedrine hydrochloride and pseudoephedrine sulphate are particularly preferred. The invention likewise encompasses mixtures of the substances mentioned.

The basic medicinally active substance is employed in therapeutically effective amounts. Preference is given to pseudoephedrine hydrochloride or pseudoephedrine-sulphate in a dosage of from 10 to 100 mg, particularly preferably in a dosage of from 20 to 70 mg.

The effervescent preparation comprises where appropriate one or more further active ingredients such as, for example, analgesics, antibiotics, antihistamines, antidepressants, antidiabetics, antihypertensives, anticoagulants, lipid-lowering agents, antineoplastics, antiviral substances, antiinflammatory substances, antitussives, expectorants, muscle relaxants, anticonvulsants, antidiarrhoeals, antiasthmatics and antidiuretics. Analgesics may be mentioned as preferred.

The analgesics include, for example, acetylsalicylic acid, paracetamol, ibuprofen, naproxen, diclofenac, propyphenazone, metamizole and COX-2 inhibitors such as, for example, celecoxib and rofecoxib. Representative antibiotics are, for example, beta-lactam antibiotics, chloramphenicol, neomycin, tetracyclines, cephalosporins, erythromycin, ciprofloxacin, moxifloxacin, norfloxacin and enrofloxacin. Representative antihistamines are, for example, fexofenadine, dimethindene, cromoglicic acid, cetirizine, loratadine, pyrilamine, chlorphenamine, tetrahydrozoline and antazoline. Representative antidepressants are, for example, amitriptyline, fluoxetine, doxepine, maprotihne and imipramine. Representative antidiabetics are, for example, acarbose, chlorpropamide, glibenclamide and tolbutamide. Representative antihypertensives are, for example, amlodipine, nifedipine, felodipine, enalapril, ramipril, captopril, cilazapril, trandolapril, fosinopril, quinapril, moexipril, lisinopril and perindopril, losartan, candesartan, irbesartan and telmisartan. Representative anticoagulants are, for example, bishydroxycoumarin and warfarin. Representative lipid-lowering agents are, for example, pravastatin, lovastatin, simvastatin, atorvastatin, fluvastatin, itavastatin, pitavastatin, rosuvastatin and cerivastatin. Representative antineoplastics are, for example, Adriamycin, fluorouracil and methotrexate. A representative antiviral substance is, for example, acyclovir. Representative antiinflammatory substances are, for example, cortisone, hydrocortisone, betamethasone, dexamethasone and prednisolone. Representative antitussives are, for example, codeine, dihydrocodeine, dextromethorphan and clobutinol. Representative expectorants are, for example, ambroxol, acetylcysteine, bromhexine and carbocysteine. Representative muscle relaxants are, for example, diazepam, dantrolene, cyclobenzaprine and methocarbamol. Representative anticonvulsants are, for example, diphenylhydantoin and barbiturates. Representative antidiarrhoeals are, for example, loperamide and diphenoxylate. Representative antiasthmatics are, for example, theophylline, beclomethasone. and epinephrine. Representative diuretics are, for example, chlorthalidone, indapamide, bendroflumethiazide, metolazone, cyclopenthiazide, polythiazide, mefruside, xipamide, chlorothiazide and hydrochlorothiazide. The listed examples are likewise understood to include the corresponding physiologically acceptable salts. The invention likewise encompasses mixtures of the active ingredients mentioned.

Further active ingredients which may be mentioned as preferred are, for example, acetylsalicylic-acid, paracetamol, ibuprofen, diclofenac, metamizole and COX-2 inhibitors such as, for example, celecoxib and rofecoxib and the physiologically acceptable salts thereof. Acetylsalicylic acid, ibuprofen, paracetamol, naproxen and the physiologically acceptable salts thereof are particularly preferred. The invention likewise encompasses mixtures of the active ingredients mentioned.

The further active ingredient(s) present where appropriate are employed in therapeutically effective amounts. Dosages of from 0.5 to 5 mmol are preferred. Dosages of from 0.8 to 3 mmol are particularly preferred.

Preference is given to acetylsalicylic acid in a dosage of from 100 to 1000 mg, paracetamol in a dosage of from 100 to 1000 mg, ibuprofen in a dosage of from 100 to 1000 mg, naproxen in a dosage of from 100 to 1000 mg or mixtures of these active ingredients in a total dosage of from 100 to 1000 mg.

Particular preference is given to acetylsalicylic acid in a dosage of from 250 to 500 mg, paracetamol in a dosage of from 250 to 500 mg, ibuprofen in a dosage of from 100 to 300 mg, naproxen in a dosage of from 250 to 500 mg or mixtures of these active ingredients in a total dosage of from 200 to 500 mg.

A preferred effervescent preparation is characterized in that an effervescent composition, pseudoephedrine and one or more analgesics such as, for example, acetylsalicylic acid, paracetamol, naproxen and ibuprofen and/or one or more antitussives such as, for example, dextromethorphan and/or one or more expectorants such as, for example ambroxol and acetylcysteine are present, with avoidance of alkalization of the urine and thus increased accumulation of the basic medicinally active substance. Antihistamines such as, for example, chlorpheniramine can be added where appropriate. The ingredients can also be in the form of their physiologically acceptable salts.

A particularly preferred effervescent preparation is characterized in that an effervescent composition, pseudoephedrine and one or more active ingredients from the group of acetylsalicylic acid, paracetamol and ibuprofen are present, with avoidance of alkalization of the urine and thus increased accumulation of the basic medicinally active substance, and it being possible for the ingredients also to be in the form of their physiologically acceptable salts.

The effervescent composition reacts on contact with water or saliva to evolve CO₂ and consists of at least one basic and of at least one acidic component.

Carbonates may be mentioned as basic component of the effervescent composition. Carbonates mean for the purposes of the invention carbonates, sesquicarbonates and bicarbonates. Preference is given to ammonium carbonate, ammonium bicarbonate and alkalimetal and alkaline earth metal carbonates and bicarbonates such as, for example, sodium carbonate, sodium bicarbonate, potassium carbonate, potassium bicarbonate, calcium carbonate, calcium bicarbonate, calcium magnesium carbonate, magnesium carbonate, magnesium bicarbonate and magnesium hydroxycarbonate. Particular preference is given to sodium carbonate, sodium bicarbonate, potassium carbonate, potassium bicarbonate, calcium carbonate, calcium bicarbonate, magnesium carbonate, magnesium bicarbonate and magnesium hydroxycarbonate. It is likewise possible to employ mixtures of the basic components mentioned.

The basic component(s) of the effervescent composition are employed in a total dosage of from 1 to 20 mmol, preferably in a total dosage of from 2 to 15 mmol, particularly preferably in a total dosage of from 2 to 10 mmol. The acidic component may have more than one dissociation constant, meaning it may have more than one acidic functional group. The acidic component may be an organic or inorganic acid in the form of its anhydride, of its free acid or of its acidic salt in which, if there is a plurality of acidic functional groups, some of the protons can be replaced by a cation or cations. Examples which may be mentioned as acidic component are tartaric acid, succinic acid, malic acid, malonic acid, maleic acid, fumaric acid, adipic acid, lactic acid, glycolic acid, alpha-hydroxy acids, ascorbic acid, isoascorbic acid, glutaric acid, amino acids, phosphoric acid, diphosphoric acid, triphosphoric acid, metaphosphoric acid, pyrophosphoric acid, polyphosphoric acids, sulphuric acid, disulphuric acid and the acidic salts thereof such as, for example, potassium hydrogentartrate or sodium hydrogenphosphate, and other physiologically acceptable salts. Particular preference is given to ascorbic acid, isoascorbic acid, tartaric acid, phosphoric acid and the acidic salts thereof. It is likewise possible to employ mixtures of the acidic components mentioned.

The acidic component(s) of the effervescent composition are employed in a total dosage of from 1 to 20 mmol, preferably in a total dosage of from 2 to 15 mmol.

It is possible to employ citric acid as acidic component if the basic component of the effervescent composition consists exclusively of alkaline earth metal carbonates and/or bicarbonates such as, for example, calcium carbonate, calcium bicarbonate, magnesium carbonate, magnesium bicarbonate and magnesium hydroxycarbonate or mixtures thereof. A preferred effervescent composition comprises citric acid as acidic component and calcium carbonate as basic component. The ratio of equivalents between the equivalents of citric acid and the total of the equivalents of the alkaline earth metal carbonates and/or bicarbonates is not more than 2:1, preferably not more than 1:1.

It is possible to employ citric acid and alkali metal carbonates and/or bicarbonates if the equivalent ratio between the equivalents of citric acid and the total of the equivalents of the alkali metal carbonates and/or bicarbonates is not more than 1:3, preferably not more than 1:4, particularly preferably not more than 1:5.

If the further active ingredient(s) which are present where appropriate is an acid, the acidic active ingredient(s) such as, for example, acetylsalicylic acid, paracetamol and ibuprofen may partly or completely replace the acidic component in the effervescent composition.

Likewise preferred as effervescent composition is a combination of at least one acidic and of at least one basic component, where the equivalent ratio between the total of the equivalents of the acidic and the total of the equivalents of the basic component is from 1:1 to 3:1, preferably 1:1 to 2.5:1 and particularly preferably 1:1 to 1.5:1.

Preference is likewise given to an effervescent preparation characterized in that an effervescent composition comprising citric acid and calcium carbonate, pseudoephedrine and one or more further active ingredients from the group of acetylsalicylic acid, paracetamol and ibuprofen are present, with avoidance of alkalization of the urine and thus increased accumulation of the basic medicinally active substance, and it being possible for the ingredients also to be in the form of their physiologically acceptable salts.

The invention further relates to a medicament comprising the effervescent preparation according to the invention and at least one further excipient.

Pharmaceutical excipients familiar to the skilled person are also described for example in the following handbook: “Handbook of Pharmaceutical Excipients”, Wade, A. & Weller, P. J., American Pharmaceutical Association, Washington, 2nd edition 1994.

Further excipients which may be mentioned for example are binders, lubricants, flavourings, wetting agents, sweeteners, antifoams, diluents, colours and stabilizers.

Binders which may be mentioned for example are glycol, polyethylene glycol, dextrose, sucrose, sugar, starch, invert sugar, mannitol, cellulose, methylcellulose and derivatives thereof.

Lubricants which may be mentioned for example are magnesium stearate, stearic acid, talc, paraffin, hydrogenated castor oil, polyethylene glycol, fumaric acid, adipic acid, sodium benzoate, sodium stearylfumarate and the salts thereof. Preferred examples are the sodium, potassium, ammonium, calcium and magnesium salts of fumaric acid and of adipic acid.

Flavourings which may be mentioned for example are synthetic and natural flavourings which are suitable for food products. Preferred examples are orange flavour, lime flavour, Optarom orange, eucalyptus oil, peppermint oil, vanilla and lemon flavour. Particularly preferred examples are orange flavour, lime flavour and Optarom orange.

Wetting agents which may be mentioned for example are dioctyl sodium sulphosuccinate and sodium lauryl sulphate.

Sweeteners which may be mentioned for example are sodium saccharin, cyclamate, invert sugar and aspartame.

An antifoam which may be mentioned for example is silicone oil.

Diluents which may be mentioned for example are starch and cellulose.

Colours which may be mentioned for example are titanium dioxide, beetroot powder, beta-carotene and all colours suitable for food products.

Stabilizers which may be mentioned for example are polyvinyl alcohol, polyvinylpyrrolidone, polyethylene glycol and derivatives thereof.

Physiologically acceptable salts for the purposes of the invention may be acid addition salts of the compounds with mineral acids, carboxylic acids or sulphonic acids. Particularly preferred salts are, for example, those with hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, citric acid, fumaric acid, maleic acid or benzoic acid.

However, salts which may be mentioned are also salts with conventional bases, such as, for example, alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylamine, procain, dibenzylamine, N-methylmorpholine, debydroabietylamine, 1-ephenamine or methylpiperidine.

The invention further relates to the use of the effervescent preparation for the treatment of diseases. Depending on the active ingredients present in the effervescent preparation, the effervescent preparation is preferably used for the treatment of influenzal infections, bacterial infections, fevers, nasal catarrhs, coughs, colds or allergies.

The invention further relates to a process for producing the effervescent preparation according to the invention, characterized in that the components of the effervescent preparation are formulated in a pharmaceutical form conventional in the area of pharmaceutical preparations. Powders, granules, beads, pellets or tablets may be mentioned as preferred. Powders, granules and tablets are particularly preferred. Suitable known processes are used to produce the medicaments according to the invention.

The present invention likewise encompasses all combinations of the preferred ranges.

### Test to Determine the Accumulation Behaviour

To investigate the accumulation behaviour of a basic medicinally active substance in effervescent preparations, the basic medicinally active substance is administered in an identical therapeutically effective amount in each case in the following three preparations: preparation A as effervescent preparation to be investigated, preparation B as non-effervescent preparation and preparation C as standard effervescent preparation, where preparation B contains no effervescent components, and preparation C contains a conventional effervescent composition consisting exclusively of 600 mg of sodium bicarbonate and 1000 mg of citric acid. The three preparations are administered at the same time on each of 5 consecutive days, with the number of dosages per day depending on the basic medicinally active substance employed, and the dosage of this substance corresponding to a conventional use. After 5 days, the respective plasma levels A, B and C of the basic medicinally active substance are measured. Plasma level B reflects the intrinsic accumulation behaviour of the basic medicinally active substance. Increased accumulation of the basic medicinally active substance occurs if the difference between plasma level A and plasma level B is in an accumulation ratio of more than 1:2, preferably of more than 1:3, particularly preferably of more than 1:4 relative to the difference between plasma level C and plasma level B. The accumulation ratio is equal to the difference between plasma level A and plasma level B divided by the difference between plasma level C and plasma level B.

A preferred effervescent preparation comprises an effervescent composition, at least one basic medicinally active substance and, where appropriate, one or more further active ingredients, characterized in that the accumulation ratio is less than 1:2, preferably less than 1:3, particularly preferably less than 1:4.

### Study of the Accumulation Behaviour of Pseudoephedrine

The accumulation behaviour of pseudoephedrine as basic medicinally active substance was investigated in two different preparations. Preparation 1 contained 60 mg of pseudoephedrine HCl, 500 mg of acetaminophen and 1 tablet of Alka-Seltzer® Cold & Cough ANC 6 (containing 600 mg of citric acid and 1000 mg of sodium bicarbonate), preparation 2 contained 20 ml of Sudafed® children's nasal decongestant liquid medication (equivalent to 60 mg of pseudoephedrine HCl). In 17 consecutive single dosages, every 6 hours the two preparations were each dissolved in 120 ml of water and administered within 2 minutes to 32 test subjects in each case, so that 4 dosages were administered on each of the first to fourth day, and one dosage was administered on the fifth day. The pseudoephedrine plasma level was measured 12 hours after the last dosage and averaged 236.9 ng/ml in the group of test subjects who received preparation 1, and averaged 120.23 ng/ml in the group of test subjects who received preparation 2. The plasma levels demonstrate increased accumulation of pseudoephedrine.

### EXEMPLARY EMBODIMENTS

### EXAMPLE 1

Effervescent tablet comprising:

| | | |
|---|---|---|
| 1. Acetylsalicylic acid | 500 | mg |
| 2. Pseudoephedrine HCl | 30 | mg |
| 3. Citric acid | 1000 | mg |
| 4. Calcium carbonate | 555 | mg |
| 5. Mannitol | 803 | mg |
| 6. Sorbitol | 100 | mg |
| 7. Povidone (K30) | 0.5 | mg |
| 8. Sodium lauryl sulphate | 0.5 | mg |

### EXAMPLE 2

Effervescent tablet comprising:

| | |
|---|---|
| 1. Acetylsalicylic acid | 500 mg |
| 2. Pseudoephedrine HCl | 30 mg |
| 3. Ascorbic acid | 200 mg |
| 4. Citric acid | 800 mg |
| 5. Calcium carbonate | 555 mg |
| 6. Mannitol | 803 mg |
| 7. Sorbitol | 100 mg |
| 8. Povidone (K30) | 0.5 mg |
| 9. Sodium lauryl sulphate | 0.5 mg |

### EXAMPLE 3

Effervescent granules comprising:

| | | |
|---|---|---|
| 1. Acetylsalicylic acid | 500 | mg |
| 2. Pseudoephedrine HCl | 30 | mg |
| 3. Sucrose | 5.120 | mg |
| 4. Ascorbic acid | 30 | mg |
| 5. Tartaric acid | 750 | mg |
| 6. Sodium bicarbonate | 690 | mg |
| 7. Sodium carbonate | 150 | mg |
| 8. Clarity Premix | 2 | mg |
| 9. Orange flavour | 30 | mg |

### EXAMPLE 4

Effervescent tablet comprising:

| | |
|---|---|
| 1. Acetylsalicylic acid | 500 mg |
| 2. Pseudoephedrine HCl | 30 mg |
| 3. Ascorbic acid | 240 mg |
| 4. Sodium dihydrogenphosphate | 900 mg |
| 5. Sodium bicarbonate | 450 mg |
| 6. Sodium carbonate | 100 mg |

### EXAMPLE 5

Effervescent granules comprising:

| | |
|---|---|
| 1. Ibuprofen | 200 mg |
| 2. Pseudoephedrine HCl | 30 mg |
| 3. Citric acid | 400 mg |
| 4. Calcium carbonate | 200 mg |
| 5. Aspartame | 13 mg |
| 6. Orange flavour | 100 mg |
| 7. Lime flavour | 20 mg |
| 8. Optarom Orange* | 200 mg |

*Lemonade base for taste optimization

### EXAMPLE 6

Effervescent tablet comprising:

| | |
|---|---|
| 1. Acetylsalicylic acid | 250 mg |
| 2. Paracetamol | 250 mg |
| 3. Pseudoephedrine sulphate | 30 mg |
| 4. Citric acid | 800 mg |
| 5. Calcium carbonate | 400 mg |

### EXAMPLE 7

Effervescent granules comprising:

| | |
|---|---|
| 1. Acetylsalicylic acid | 500 mg |
| 2. Pseudoephedrine HCl | 30 mg |
| 3. Citric acid | 180 mg |
| 4. Sodium dihydrogenphosphate | 900 mg |
| 5. Sodium bicarbonate | 450 mg |
| 6. Sodium carbonate | 100 mg |

### EXAMPLE 8

Effervescent tablet comprising:

| | |
|---|---|
| 1. Acetylsalicylic acid | 500 mg |
| 2. Pseudoephedrine HCl | 30 mg |
| 3. Sodium carbonate | 280 mg |

### EXAMPLE 9

Effervescent tablet comprising:

| | |
|---|---|
| 1. Pseudoephedrine HCl | 30 mg |
| 2. Citric acid | 2882 mg |
| 3. Calcium carbonate | 750 mg |

### EXAMPLE 10

Effervescent tablet comprising:

| | |
|---|---|
| 1. Pseudoephedrine HCl | 30 mg |
| 2. Ascorbic acid | 1321 mg |
| 3. Sodium bicarbonate | 630 mg |

1. Effervescent preparation comprising at least one basic medicinally active substance, characterized in that increased accumulation of the basic medicinally active substance is avoided.

2. Effervescent preparation according to claim 1, characterized in that the accumulation ratio is less than 1:2.

3. Effervescent preparation according to either of claims 1 to 2, characterized in that at least one substance from the group of pseudoephedrine, chlorphenamine, phenylephrine, clemastine and the physiologically acceptable salts thereof is present as basic medicinally active substance.

4. Effervescent preparation according to any of claims 1 to 3, characterized in that pseudoephedrine, pseudoephedrine hydrochloride or pseudoephedrine sulphate is present as basic medicinally active substance.

5. Effervescent preparation according to any of claims 1 to 4, characterized in that one or more further active ingredients are present.

6. Effervescent preparation according to claim 5, characterized in that analgesics are present as one or more further active ingredients.

7. Effervescent preparation according to claim 5, characterized in that at least one substance from the group of acetylsalicylic acid, paracetamol, ibuprofen, diclofenac, metamizole, celecoxib, rofecoxib and the physiologically acceptable salts thereof is present as further active ingredient.

8. Effervescent preparation according to claim 5, characterized in that acetylsalicylic acid in a dosage of from 250 to 500 mg, paracetamol in a dosage of from 250 to 500 mg, ibuprofen in a dosage of from 100 to 300 mg, naproxen in a dosage of from 250 to 500 mg or mixtures of these substances in a total dosage of from 200 to 500 mg are present as further active ingredient.

9. Effervescent preparation according to claim 4, characterized in that an effervescent composition, pseudoephedrine and at least one further active ingredient from the group of acetylsalicylic acid, paracetamol, naproxen, ibuprofen, dextromethorphan, ambroxol, acetylcystein and the physiological acceptable salts thereof are present.

10. Effervescent preparation according to claim 4, characterized in that an effervescent composition, pseudoephedrine and at least one further active ingredient from the group of acetylsalicylic acid, paracetamol, naproxen, ibuprofen, dextromethorphan, ambroxol, acetylcystein, chlorphenamine and the physiological acceptable salts thereof are present.

11. Effervescent preparation according to claim 10, characterized in that an effervescent composition, pseudoephedrine-HCl and acetylsalicylic acid and/or ibuprofen are present.

12. Effervescent preparation according to any of claims 1 to 11, characterized in that sodium carbonate, sodium bicarbonate, potassium carbonate, potassium bicarbonate, calcium carbonate, calcium bicarbonate, magnesium carbonate, magnesium bicarbonate, magnesium hydroxycarbonate or a mixture of these substances is present as basic component of the effervescent composition.

13. Effervescent preparation according to any of claims 1 to 12, characterized in that ascorbic acid, isoascorbic acid, tartaric acid, phosphoric acid, the acidic salts thereof or a mixture of these substances is present as acidic component of the effervescent composition.

14. Effervescent preparation according to any of claims 1 to 13, characterized in that citric acid is present as acidic component, and exclusively alkaline earth metal carbonates or bicarbonates or mixtures thereof are present as basic component of the effervescent composition.

15. Effervescent preparation according to claim 15, characterized in that citric acid is present as acidic component, and exclusively alkaline earth metal carbonates or bicarbonates or mixtures thereof are present as basic component of the effervescent composition, where the equivalent ratio between the equivalents of citric acid and the total of the equivalents of the alkaline earth metal carbonates and/or bicarbonates is not more than 2:1.

16. Effervescent preparation according to any of claims 1 to 15, characterized in that citric acid is present as acidic component of the effervescent composition, and alkali metal carbonates and/or bicarbonates are present as basic component of the effervescent composition, where the equivalent ratio between the equivalents of citric acid and the total of the equivalents of the alkali metal carbonates and/or bicarbonates is not more than 1:3.

17. Effervescent preparation according to either of claims 14 to 15, characterized in that pseudoephedine-HCl, acetylsalicylic acid, citric acid and calcium carbonate are present.

18. Effervescent preparation according to any of claims 1 to 12, characterized in that at least one further active ingredient is an acid which partly or completely replaces the acidic component of the effervescent composition.

19. Effervescent preparation according to any of claims 1 to 18, characterized in that the equivalent ratio between the total of the equivalents of the acidic and the total of the equivalents of the basic component is from 1:1 to 3:1.

20. Medicament comprising an effervescent preparation as defined in any of claims 1 to 19, and at least one further excipient.

21. Medicament according to claim 20 in the form of powder, granules, beads, pellets or tablets.

22. Use of an effervescent preparation as defined in any of claims 1 to 19 for producing a medicament for the treatment of diseases.

23. Process for producing a medicament as defined in claims 20 or 21, characterized in that the individual components are formulated and processed to powder, granules, beads, pellets or tablets.
